SEQUENCE LISTING (8 sequences)

SEQ ID NO: 1 - DNA, 24 bp, INDUCIBLE NITRIC OXIDE SYNTHASE
catggcttgcccctggaagtttct

SEQ ID NO: 2 - DNA, 23 bp, INDUCIBLE NITRIC OXIDE SYNTHASE
cctctatggtgccatcgggcatc

SEQ ID NO: 3 - DNA, 22 bp, ARGINASE
ctctaagggacagcctcgagga

SEQ ID NO: 4 - DNA, 23 bp, ARGINASE
tgggttcacttccatgatatcta

SEQ ID NO: 5 - DNA, 27 bp, ARGINASE II
atgtccctaaggggcagcctctcgcgt

SEQ ID NO: 6 - DNA, 27 bp, ARGINASE II
cacagctgtagccatctgacacagctc

SEQ ID NO: 7 - DNA, 21 bp, INDUCIBLE NITRIC OXIDE SYNTHASE
ccagctagccaaagtcaccat

SEQ ID NO: 8 - DNA, 21 bp, INDUCIBLE NITRIC OXIDE SYNTHASE
gtctcggagccatacaggatt

(19) United States
(12) Patent Application Publication
(10) Pub. No.: US 2007/0219208 A1
(43) Pub. Date: Sep. 20, 2007

(54) Methods for Treating Cancer

(76) Inventors: Balaraman Kalyanaraman, Milwaukee, WI (US); Srigiridhar Kotamraju, Hyderabad (IN)

Correspondence Address: QUARLES & BRADY LLP, 411 E. WISCONSIN AVENUE, SUITE 2040, MILWAUKEE, WI, 53202-4497, US

(21) Appl. No.: 11/677,730

(22) Filed: Feb. 22, 2007

Related U.S. Application Data
(60) Provisional application No. 60/777,041, filed on Feb. 27, 2006.

(51) Int. Cl. A61K 31/525; A61K 31/401; A61K 31/366; A61K 31/22; A61K 31/198

(52) U.S. Cl. 514/250; 514/423; 514/460; 514/548; 514/565; 514/251

(57) ABSTRACT

It is disclosed here that HMG-CoA reductase inhibitors inhibit the proliferation and cause the death of breast cancer cells by inducing the expression of inducible nitric oxide synthase (iNOS) to promote intracellular nitric oxide formation, which the inventors found to be accomplished through the inhibition of protein geranylgeranylation. The disclosure here enables a new breast cancer treatment strategy that combines the inhibition HMG-CoA reductase or protein geranylgeranylation and the promotion of nitric oxide formation by iNOS.

### CROSS-REFERENCE TO RELATED APPLICATION

This application claims the benefit of U.S. provisional application 60/777,041, filed on Feb. 27, 2006, which is incorporated by reference in its entirety.

### STATEMENT REGARDING FEDERALLY SPONSORED RESEARCH OR DEVELOPMENT

This invention was made with United States government support awarded by the following agency: NIH HL-067244. The United States has certain rights in this invention.

### BACKGROUND OF THE INVENTION

Statins are widely used, FDA-approved cholesterol-lowering drugs. Statins selectively inhibit the enzyme hydroxymethylglutaryl coenzyme A (HMG-CoA) reductase and cholesterol biosynthesis. Recent data suggest that statins can also prevent various types of cancers (e.g., breast, skin, and colorectal cancers) and stimulate apoptotic cell death in various types of tumor cells (e.g., leukemia, lymphoma, and neuroblastoma cells). Currently, the National Cancer Institute is sponsoring clinical trials to evaluate the efficacy of statins in the treatment of colorectal and skin cancers. However, the exact mechanisms by which statins kill cancer cells are not known. Understanding the cancer cell killing mechanism of statins may provide new tools for cancer prevention and therapy.

### SUMMARY OF THE INVENTION

It is disclosed here that HMG-CoA reductase inhibitors inhibit the proliferation and cause the death of breast cancer cells by inducing or stimulating the expression of inducible nitric oxide synthase (iNOS) and augmenting intracellular nitric oxide formation, which the inventors found to be accomplished through the inhibition of HMG-CoA reductase and downstream protein geranylgeranylation. The disclosure here enables a new breast cancer treatment strategy that combines the inhibition HMG-CoA reductase or protein geranylgeranylation and the promotion of nitric oxide formation by iNOS.

### BRIEF DESCRIPTION OF THE DRAWINGS

FIG. 1 shows the effects of statin and mevalonate on cell death and cell proliferation in MCF-7 and MCF-10A cells. A: MCF-7 cells were treated with simvastatin or fluvastatin (5-10 μM) for 24-48 h and cell death was measured by the 3-(4,5-dimethylthiazol-2-yl)-2,5-diphenyltetrazolium bromide (MTT) assay. B: MCF-7 and MCF-10A cells were treated with simvastatin or fluvastatin (5-10 μM) for a period of 48 h and cell death was measured by the MTT assay. C: The effect of mevalonate (20 μM) on cell death induced by simvastatin and fluvastatin as measured by the MTT assay. D and E: The effect of varying concentrations of simvastatin and fluvastatin in the presence or absence of mevalonate (20 μM) on cell proliferation as measured by ³H-thymidine uptake into cells after a 48 h treatment. Data represent the mean±SD from three different experiments. *, significantly different (p<0.05) compared with untreated conditions and #, significantly different (p<0.05) compared to simvastatin or fluvastatin alone.

FIG. 2 shows the effects of statins and mevalonate on nitric oxide generation, arginase levels and cell death in MCF-7 cells. A: Cells were treated with simvastatin or fluvastatin (10 μM) in the presence or absence of mevalonate (20 μM) for 40 h and intracellular NO was measured by DAF fluorescence as described in “Materials and Methods” below. The fluorescence intensity was calculated using the Metamorph Image analysis software. B-D: Inducible NOS mRNA was measured by RT-PCR (B), protein levels measured by Western analysis (C) and NO₂⁻/NO₃⁻ levels (D) were measured as described in “Materials and Methods.” Cells were treated with simvastatin and fluvastatin (5-20 μM) for 40 h in the presence and absence of mevalonate (20 μM). D: MCF-7 cells were treated with simvastatin or fluvastatin (10 μM) for 40 h in the presence or absence of mevalonate (20 μM) and RT-PCR was performed using the gene specific primers for measuring arginase II transcript levels. F: MCF-7 cells were treated with varying concentrations of fluvastatin or NO-fluvastatin (0-1 μM) for a period of 48 h and cell death was analyzed by the MTT assay. Data represent the mean±SD of three independent experiments. *, significantly different (p<0.05) compared with untreated conditions and #, significantly different (p<0.05) compared to simvastatin or fluvastatin alone.

FIG. 3 shows the effects of geranylgeranyl transferase inhibitor (GGTI-298) and farnesyl transferase inhibitor (FTI-277) on cell death, cell proliferation and NO levels in MCF-7 cells. A: Cells were treated with GGTI or FTI (10-20 μM) for a period of 48 h and cell death was measured by the MTT assay. B: Conditions same as (A) but cell proliferation was measured using the ³H-thymidine uptake as described in “Materials and Methods.” C: MCF-7 cells were treated with GGTI or FTI (10-20 μM) for 40 h and iNOS protein levels were measured by the Western analysis. D: Same as (A) except that NO₂⁻/NO₃⁻ levels were measured at the end of the experiment using the NO analyzer. Data represent the mean±SD of three independent experiments. *, significantly different (p<0.05) compared with untreated conditions and #, significantly different (p<0.05) compared to FTI treatment alone.

FIG. 4 shows the effects of 1400 W, sepiapterin and mevalonate on statin-induced cell death and NO levels in MCF-7 cells. A: Cells were treated with simvastatin or fluvastatin (10 μM) in the presence or absence of a specific iNOS inhibitor, 1400 W (10 μM) for 48 h and cell death was measured by the MTT assay. B: Same as (A) except that cells were also treated with statins in the presence or absence of sepiapterin (50 μM) for 40 h and NO₂⁻/NO₃⁻ levels were measured using the NO analyzer. Data represent the mean±SD of at least three independent experiments. *, significantly different (p<0.05) compared with untreated conditions and #, significantly different (p<0.05) compared to simvastatin or fluvastatin alone.

FIG. 5 shows the effects of 1400 W and mevalonate on statin-induced cell cycle protein alterations in MCF-7 cells. A (Table): The cell cycle distribution of MCF-7 cells treated with either simvastatin or fluvastatin (5-10 μM) for 40 h in the presence or 1400 W (10 μM) or mevalonate (20 μM). The cell sorting was performed by flow cytometry as described in “Materials and Methods.” B: Cells were treated with simvastatin or fluvastatin (10 μM) in the presence or absence of 1400 W (10 μM) or mevalonate (20 μM) for 40 h and cyclins D1 and E protein levels were measured by the Western analysis using the corresponding polyclonal or monoclonal antibodies. Data are representative of three separate experiments.

FIG. 6 shows the effects of 1400 W, sepiapterin and mevalonate on statin-induced caspase-3 like activity, DNA fragmentation and their clonogenic abilities in soft agar. A: The caspase-3 like proteolytic activity was measured in MCF-7 cells treated with simvastatin or fluvastatin (10 μM) for 48 h in the presence or absence of 1400 W (10 μM), mevalonate (20 μM) or sepiapterin (50 μM). Cell lysates were incubated with the fluorogenic caspase-3 substrate (DEVD-AFC) for 1 h at 37° C. and the released fluorescent active product was measured in a fluorescence spectrophotometer using an excitation/emission of 400/505 nm, respectively. B: Images of anchorage-independent colony formation of MCF-7 cells treated simvastatin or fluvastatin (10 μM) in the presence or absence of mevalonate (20 μM), 1400 W (10 μM) or sepiapterin (50 μM). Cells were also treated with either GGTI-298 or FTI-277 (10 μm) alone. Treatments were carried out with the above mentioned conditions for 40 h and seeded onto soft agar plates as described in “Materials and Methods.” After 21 days, colonies were stained with 0.005% Crystal violet and viewed under 10× magnification and colonies were counted manually. Data represent the mean±SD measured from at least three different experiments. *, significantly different (p<0.05) compared with untreated conditions.

### DETAILED DESCRIPTION OF THE INVENTION

The present invention is based on the inventors' discovery that HMG-CoA reductase inhibitors inhibit the proliferation and cause the death of breast cancer cells by inducing the expression of inducible nitric oxide synthase (iNOS) and inhibiting the expression of arginase, leading to an increase in the level of nitric oxide (.NO or NO) in breast cancer cells. The discovery provides new tools for treating breast cancer in that an HMG-CoA reductase inhibitor can now be used together with an agent that can enhance the iNOS-catalyzed NO formation to more effectively treat breast cancer. This has been demonstrated by the inventors using the HMG-CoA reductase inhibitor simvastatin or fluvastatin in combination with sepiapterin, a precursor to the iNOS cofactor/activator 5,6,7,8-tetrahydrobiopterin (5,6,7,8-BH₄) for catalyzing NO formation. It is envisioned other methods of increasing the level of BH₄ and other methods of increasing NO formation by iNOS can also be used. The inventors further discovered that the above effects of HMG-CoA reductase inhibitors on breast cancer cells and iNOS expression are achieved through inhibiting protein geranylgeranylation. Therefore, similar to HMG-CoA reductase inhibitors, protein-geranylgeranylation inhibitors can be used together with an agent that can enhance the iNOS-catalyzed NO formation to more effectively treat breast cancer.

In one aspect, the present invention relates to a method for treating breast cancer in a human or non-human animal (e.g., a mammal) by administering to a human or non-human animal in need of said treatment a first agent selected from an HMG-CoA reductase inhibitor, an HMG-CoA reductase inhibitor coupled with a nitric oxide molecule, a protein geranylgeranyl transferase (GGTase) inhibitor, and a GGTase inhibitor coupled with a nitric oxide molecule and a second agent that promotes iNOS-catalyzed nitric oxide formation wherein the amount of the first agent and the amount of the second agent are therapeutically effective. The method may optionally include a step of evaluating the effectiveness of the treatment by monitoring the size of the malignant breast tissue or tumor. A slow down in tumor size increase, a stabilization of the tumor size, or a decrease in the size of the tumor indicates that the treatment is effective.

In another aspect, the present invention relates to a method for inhibiting the proliferation or causing the death of breast cancer cells of a human or non-human animal (e.g., a mammal) by exposing the cells to a first agent selected from an HMG-CoA reductase inhibitor, an HMG-CoA reductase inhibitor coupled with a nitric oxide molecule, a GGTase inhibitor, and a GGTase inhibitor coupled with a nitric oxide molecule and a second agent that promotes iNOS-catalyzed nitric oxide formation wherein the amount of the first agent and the amount of the second agent are sufficient to inhibit the proliferation or cause the death of breast cancer cells. By breast cancer cells, we mean cells that are located either in vivo (including cells in situ and transplanted cells) or in vitro (e.g., in culture), which can include cells of breast cancer and mammary carcinoma cell lines. The method may optionally include a step of monitoring the proliferation inhibition and the death of the breast cancer cells. For an in vivo application, this may involve monitoring the size of the malignant breast tissue or tumor.

In another aspect, the present invention relates to a method for treating breast cancer in a human or non-human animal (e.g., a mammal) by administering to a human or non-human animal in need of said treatment an agent selected from an HMG-CoA reductase inhibitor coupled with a nitric oxide molecule and a GGTase inhibitor coupled with a nitric oxide molecule wherein the amount of the agent is therapeutically effective. The method may optionally include a step of evaluating the effectiveness of the treatment by monitoring the size of the malignant breast tissue or tumor. A slow down in tumor size increase, a stabilization of the tumor size, or a decrease in the size of the tumor indicates that the treatment is effective.

HMG-CoA reductase inhibitors, also referred to as statins, are well known in the art. Examples of known inhibitors include lovastatin, simvastatin, pravastatin, fluvastatin, atorvastatin, mevastatin, cerivastatin, pitavastatin, rosuvastatin, compactin, dalvastatin, and fluindostatin. In one embodiment, a hydrophobic (insoluble in water) statin, such as lovastatin, simvastatin, fluvastatin, atorvastatin, mevastatin, cerivastatin, pitavastatin, rosuvastatin, compactin, or dalvastatin, is used to practice the present invention. In another embodiment, simvastatin or fluvastatin is used.

Protein geranylgeranyl transferase (GGTase), also referred to as protein geranylgeranyl transferase I (GGTase I), adds a geranylgeranyl group to proteins bearing a CaaX motif. Any known GGTase inhibitor, including GGTase-specific inhibitors and those that inhibit both GGTase and farnesyl-protein transferase (FPTase), can be used to practice the present invention. Examples of known GGTase inhibitors include those described in U.S. Pat. No. 5,470,832, U.S. Pat. No. 5,965,539 and U.S. Pat. No. 6,586,461, GGTI 297 and GGTI 298 disclosed by T. F. McGuire et al. (J Biol Chem 271:24702-24707, 1996), GGTI-286 and GGTI-287 that are commercially available from Calbiochem-Novabiochem Corporation (La Jolla, Calif.), Massadine (Nishimura et al., Org Lett. 5:2255-7, 2003), and Candida albicans GGTase inhibitors (see e.g., Murthi, et al., Bioorg Med Chem Lett 13:1935-7, 2003; and Sunami et al., Bioorg Med Chem Lett 12:629-32, 2002).

Examples of known non-selective FPTase/GGTase inhibitors include those described in Nagasu et al. (Cancer Res 55:5310-5314, 1995; and PCT application WO 95/25086).

By “a HMG-CoA reductase inhibitor coupled with a nitric oxide molecule,” we mean a hybrid molecule containing a nitric oxide releasing moiety combined with a statin. Likewise, by “a GGTase inhibitor coupled with a nitric oxide molecule,” we mean a hybrid molecule containing a nitric oxide releasing moiety combined with a GGTase inhibitor. It is well within the capability of a skilled artisan to make such hybrid molecules. N-nitroso-fluvastatin (NO-fluvastatin) is an example (Ongini E et al. Proc Natl Acad Sci USA 101:8497-8502, 2004).

Any agent that can promote nitric oxide formation by iNOS can be used to practice the present invention. Examples of such agents include endogenous iNOS cofactor/activator BH₄ and synthetic NOS activators, compounds that can be converted to BH₄ intracellularly, compounds that facilitate the regeneration of BH₄ intracellularly, iNOS substrate L-arginine for nitric oxide formation and compounds that can be converted to L-arginine intracellularly, arginase inhibitors, and compounds that can increase the metabolism of asymmetric dimethyl-arginine (ADMA).

iNOS catalyzes the formation of nitric oxide from L-arginine. This process requires the presence of its natural cofactor/activator 5,6,7,8-BH₄. 5,6,7,8-BH₄ is generated inside a cell via its de novo synthesis pathway using GTP as a precursor (see e.g., Gross S S et al. J Biol Chem 267:25722-25729, 1992; and Thony B et al. Biochem J 347:1-16, 2000). 5,6,7,8-BH₄ is also generated inside a cell through a salvage pathway in which sepiapterin is converted first to 7,8-dihydrobiopterin (7,8-BH₂) and then to 5,6,7,8-BH₄. Administering BH₄ or its precursor sepiapterin has been shown to be able to restore impaired nitric oxide activity in vivo (see e.g., Stroes E et al. J Clin Invest 99:41-46, 1997; and Tiefenbacher C P et al. Circulation 102:2172-2179, 2000). Certain pteridine derivatives have been shown to be able to replace BH₄ to activate NO synthesis (see e.g., U.S. 2006/0194800).

As a cofactor of iNOS, 5,6,7,8-BH₄ is oxidized to quinoid dihydrobiopterin (qBH₂) during the formation of nitric oxide and 5,6,7,8-BH₄ is regenerated from qBH₂ by dihydropteridine reductase. Folates have been shown to stimulate 5,6,7,8-BH₄ regeneration from qBH₂ and administering the active form of folic acid 5-methyltetrahydrofolate has been shown to restore impaired nitric oxide activity in vivo (see e.g., Verhaar V C et al., Circulation 97:237-241, 1998; and Van Etten R W et al. Diabetologia 45:1004-1010, 2002).

The present invention contemplates the use of BH₄ as well as other synthetic NOS activators, which are known in the art, to increase nitric oxide formation by iNOS. In this context, the term BH₄ refers to all natural and unnatural stereoisomeric forms of tetrahydrobiopterin, pharmaceutically acceptable salts thereof and any mixtures of the isomers and the salts. Examples of synthetic NOS activators include 6-methyltetrahydropterin (see e.g., Hevel J M et al. Biochemistry 31:7160-5, 1992) and the pteridine derivatives disclosed in U.S. 2006/0194800 (see the compounds defined by formula (I)), both of which are herein incorporated by reference as if set forth in their entirety.

As used herein, the term “pharmaceutically acceptable salts” refers to salts prepared from pharmaceutically acceptable non-toxic acids, including inorganic acids and organic acids.

The present invention also contemplates the use of compounds that can be converted to 5,6,7,8-BH₄ inside a cell, such as 5,6,7,8-BH₄ precursors in its de novo synthesis pathway (e.g., 7,8-dihydroneopterin triphosphate and 6-pyruvoyl-tetrahydropterin, Scheme 1 in Thony B et al. Biochem J 347:1-16, 2000), to increase nitric oxide formation by iNOS. Other examples include sepiapterin and BH₂. In this context, the terms “sepiapterin” and “BH₂” refers to all their natural and unnatural stereoisomeric forms, pharmaceutically acceptable salts thereof and any mixtures of the isomers and the salts.

The present invention further contemplates the use of agents such as folic acid or folate that facilitates the regeneration of BH₄ inside a cell. By folate, we mean a folate compound or a folate derivative compound. The term “folate derivative compound” will be readily understood by those of skill in the art to encompass compounds having a folate “backbone” which has been derivatized. Therefore, the term folate may include, for example, one or more of the folylpolyglutamates, compounds in which the pyrazine ring of the pterin moiety of folic acid or of the folylpolyglutamates is reduced to give dihydrofolates or tetrahydrofolates, or derivatives of all the preceding compounds in which the N-5 or N-10 positions carry one carbon units at various levels of oxidation, or pharmaceutically acceptable salts thereof or a combination of two or more thereof. Examples of suitable folate and folate derivative compounds include dihydrofolate, tetrahydrofolate, 5-methyltetrahydrofolate, 5,10-methylenetetrahydrofolate, 5,10-methenyltetrahydrofolate, 5,10-formiminotetrahydrofolate, 5-formyltetrahydrofolate (leucovorin), 10-formyltetrahydrofolate, 10-methyltetrahydrofolate, pharmaceutically acceptable salts thereof, or a combination of two or more thereof. 5-methyltetrahydrofolic acid and 5-methyltetrahydrofolate are preferred compounds for the purpose of the present invention.

The present invention also contemplates the use of arginine such as the endogenous iNOS substrate L-arginine or a derivative thereof to promote nitric oxide formation. As used herein, the tern “arginine” or “L-arginine” refers to arginine or L-arginine and all of its biochemical equivalents, e.g., arginine hydrochloride or L-arginine hydrochloride, precursors, and its basic form, that act as substrates of NOS with resulting increase in production of nitric oxide. The term includes pharmaceutically acceptable salts of arginine and L-arginine such as arginine hydrochloride, arginine aspartate, or arginine nicotinate. Other suitable arginine compounds or derivatives may be chosen from di-peptides that include arginine such as alanylarginine (ALA-ARG), valinyL-arginine (VAL-ARG), isoleucinyL-arginine (ISO-ARG), and leucinyL-arginine (LEU-ARG), and tri-peptides that include arginine such as argininyl-lysinyl-glutamic acid (ARG-LYS-GLU) and arginyl-glysyL-arginine (ARG-GLY-ARG).

Another way to make more L-arginine available for nitric oxide synthesis by iNOS is to inhibit the activity of arginase. In addition to iNOS, L-arginine is also a substrate of arginases which converts L-arginine to L-ornithine and urea. Inhibiting the activity of arginase will make more L-arginine available for nitric oxide formation by iNOS. Any arginase inhibitor known in the art can be used to practice the present invention. Examples of the inhibitors include N-hydroxy-L-arginine (see e.g., Chenais et al. Biochem Biophys Res Commun 196:1558-1565, 1993; and Daghigh et al. Biochem Biophys Res Commun 202:174-180, 1994) and those described in U.S. 20030036529, which is herein incorporated by reference in its entirety. One class of arginase inhibitors disclosed in U.S. 20030036529, including S-(2-boronoethyl)-L-cysteine (BEC) and 2(S)-amino-6-boronohexanoic acid (ABHA), has the structure of HOOC—CH(NH₂)—X¹—X²—X³—X⁴—B(OH)₂, wherein each of X¹, X², X³, and X⁴ is selected from the group consisting of —(CH₂)—, —S—, —O—, —(NH)—, and —(N-alkyl)-. In one subclass, X² is not —S— when each of X¹, X³, and X⁴ is —(CH)₂—.

Asymmetric dimethyl-arginine (ADMA) is an endogenous, competitive inhibitor of NOS and therefore the present invention also contemplates the use of an agent that can increase the metabolism of ADMA to promote nitric oxide formation by iNOS. Examples of such agents include compounds that facilitate the formation or enhancement of the activity of the intracellular enzyme dimethylarginine dimethylaminohydrolase responsible for degradation of ADMA or inhibitors of S-adenosylmethionine-dependent methyltransferase that is responsible for formation of ADMA (Matsuguma K et al., J Am Soc Nephrol 8:2176-83, 2006, which is herein incorporated by reference in its entirety).

The first agent and the second agent can be administered or used to contact breast cancer cells simultaneously or sequentially (e.g., the first agent followed by the second agent). When administered separately, each agent is administered with a pharmaceutically acceptable carrier. When administered or used simultaneously, the two agent can be provided in one composition or two separate compositions and the compositions can further contain a pharmaceutically acceptable carrier.

As used herein, the term “pharmaceutically acceptable carrier” means a carrier medium which does not interfere with the effectiveness of the biological activity of the active ingredient and which is not toxic to the subject to which it is administered. The use of such media for pharmaceutically active formulations is well known in the art.

In another aspect, the present invention relates to a composition that contains a first agent as described above, a second agent as described above, and a pharmaceutically acceptable carrier wherein the amount of the first agent and the amount of the second agent are pharmaceutically effective for treating breast cancer. In one embodiment, the first agent is an HMG-CoA reductase inhibitor or an HMG-CoA reductase inhibitor coupled with a nitric oxide molecule. In another embodiment, the first agent is a GGTase inhibitor or a GGTase inhibitor coupled with a nitric oxide molecule. In some embodiments, the second agent is sepiapterin. In some other embodiments, the second agent is 6-methyltetrahydrobpterin or 6-pyruvonyl tetrahydropterin. In still some other embodiments, the second agent is folic acid or folate.

In another aspect, the present invention relates to a kit that contains a first agent as described above, a second agent as described above, and an instruction manual on administering the agents to treat breast cancer according to the method provided herein wherein the amount of the first agent and the amount of the second agent are pharmaceutically effective for treating breast cancer. In this regard, the first agent and the second agent can be provided in separate compositions or one single composition. In one embodiment, the first agent is an HMG-CoA reductase inhibitor or an HMG-CoA reductase inhibitor coupled with a nitric oxide molecule. In another embodiment, the first agent is a GGTase inhibitor or a GGTase inhibitor coupled with a nitric oxide molecule. In some embodiments, the second agent is sepiapterin. In some other embodiments, the second agent is 6-methyltetrahydrobpterin or 6-pyruvonyl tetrahydropterin. In still some other embodiments, the second agent is folic acid or folate.

The invention will be more fully understood upon consideration of the following example, which is not intended to limit the scope of the invention.

### EXAMPLE

This example shows that (i) statins diminish proliferation and promote apoptosis in MCF-7 breast cancer cells but not non-cancerous MCF-10 epithelial cells through elevation of inducible NOS expression and NO formation from oxidation of L-arginine to L-citruline using 5,6,7,8-BH₄ as a co-factor, (ii) supplementation with sepiapterin, a precursor to 5,6,7,8-BH4 biosynthesis, enhanced statin-mediated proapoptotic and anti-proliferative effects in MCF-7 cells, (iii) statin-mediated tumoricidal effects occur through inhibition of geranylgeranyl transferase inhibition, not farnesyl transferase.

In particular, this example shows that statin treatment enhanced the caspase-3 like activity and DNA fragmentation in MCF-7 cells, and significantly inhibited MCF-7 cell proliferation but not MCF-10 cells (non-cancerous epithelial cells). Statin-induced cytotoxic effects were reversed by mevalonate, an immediate metabolic product of acetyl CoA/HMG-CoA reductase reaction. Both simvastatin and fluvastatin induced nitric oxide (.NO) as measured by DAF-2T formation and NO₂⁻/NO₃⁻ levels. Statin-induced .NO and tumor cell cytotoxicity were inhibited by 1400 W, a more specific inhibitor of inducible nitric oxide synthase (iNOS or NOS 11). Both fluvastatin and simvastatin increased iNOS mRNA and protein expression. Mevalonate inhibited statin-induced iNOS and .NO. Stimulation of iNOS by statins via inhibition of geranylgeranylation by GGTI-298 but not farnesylation by FTI-277 enhanced the proapoptotic effects of statins in MCF-7 cells. Statin-mediated antiproliferative and proapoptotic effects were exacerbated by sepiapterin, a precursor of tetrahydrobiopterin, an essential co-factor of NO biosynthesis by NOS. Therefore, iNOS-mediated .NO is responsible for the proapoptotic, tumoricidal, and antiproliferative effects of statins in MCF-7 cells.

Materials and Methods

Reagents, Cell Lines and Culture Conditions:

Simvastatin, fluvastatin, N-4-[2(R)-amino-3-mercaptopropyl]amino-2-naphthylbenzoyl-(L)-leucine methyl ester (GGTI-298), methyl {N-[2-phenyl-4-N[2(R)-amino-3-mecaptopropylamino]benzoyl]}-methionate (FTI-277), 4,5-diaminofluorescein Diacetate (DAF-2-DA) were purchased from Calbiochem (La Jolla, Calif.). Mevalonate, N-(3-aminomethyl)benzylacetamidine (1400 W), [3-(4,5-dimethylthiazol-2-yl)-2,5-diphenyltetrazolium bromide] (MTT), squalene and sepiapterin were purchased from Sigma Inc. (St. louis, Mo.). NO-fluvastatin (NCX 6553) was from Cayman Chemicals (Ann Arbor, Mich.). The culture medium (MEM) and fetal bovine serum were from Life Technologies, Inc. (Grand Island, N.Y.). All other chemicals were of reagent grade. All cell lines were purchased from the American Type Culture Collection (Rockville, Md.).

MCF-7 and MDA-MB-231 cells were grown in 10% minimum essential medium (MEM) containing 10% FBS, L-glutamine (4 mmol/L), penicillin (100 units/ml), and streptomycin (100 μg/ml), and incubated at 37° C. in a humidified atmosphere of 5% CO₂ and 95% air.

MTT Reduction Cytotoxicity Assay:

MTT is taken up by cells and is reduced to a colored formazon product that can be detected by spectrophotometry (max=562 nm). Reduction of MTT is dependent upon the mitochondrial respiratory function, and thus measures the relative number of viable cells in the culture. After the treatment was completed, MCF-7 cells were washed twice with DPBS and taken in a ml of MEM without FBS and incubated with 5 mg/ml MTT solution for 1 h at 37° C. Medium was removed and cells were solubilized in DMSO. The absorption was measured at 562 nm with reference at 630 nm.

Thymidine Uptake Studies:

DNA synthesis was measured by monitoring the uptake of tritiated thymidine, [³H]TdR (Perkin-Elmer, Boston, Mass.). Cells (5×10⁵/ml) were cultured with different concentrations of simvastatin or fluvastatin (0-10 μM) in the presence or absence of mevalonate (20 μM), 1400 W, or sepiapterin. Cells were pulse-chased with [³H]TdR [0.5 μCi (0.185 MBq)/well during the last 3 h of a 24 h culture, harvested onto glass filters with an automatic cell harvester (Cambridge Technology, Cambridge, Mass.), and counted using the LKB Betaplate scintillation counter (Wallac, Gaithersburg, Md.). All experiments were performed in triplicate and repeated three times.

Measurement of Intracellular .NO:

Intracellular .NO levels were monitored using a DAF-2-DA fluorescence probe (Rodriguez J, et al. Free Radic Biol Med, 38:356-68, 2005). After the treatments, cells were washed with DPBS and incubated in 2 ml of fresh culture medium without FBS. DAF-2-DA was added at a final concentration of 10 μM, and cells were incubated for 20 min. Cells were washed twice with DPBS and maintained in 1 ml of the culture medium for monitoring the fluorescence using a Nikon fluorescence microscope (excitation, 488 nm; emission, 610 nm) equipped with an FITC filter. Fluorescence intensity was calculated using the Metamorph software.

Nitrite and Nitrate Measurements:

Nitrite and nitrate, the oxidative metabolites of .NO, were measured by chemiluminescence, using the Sievers' apparatus, following reduction with vanadium (III) chloride (Pritchard K A, Jr, et al. J Biol Chem; 276:17621-4; 2001). Briefly, following treatments, cells were washed three times with DPBS after aspirating the medium. To this, 1 ml of Hanks' balanced salt mixture containing 25 μM L-arginine was added and incubated for 30 min at 37° C. The medium was collected and centrifuged for 5 min at 5000 rpm, and 50 μl of the clear supernatant was used for nitrate and nitrite analysis. Each sample was analyzed in triplicate.

Western Blot Analysis:

After treatment with statins, cells were washed with ice-cold DPBS and resuspended in 150 μl of radioimmune precipitation assay buffer (20 mM Tris-HCl, pH 7.4, 2.5 mM EDTA, 1% Triton X-100, 1% sodium deoxycholate, 1% SDS, 100 mM NaCl, 100 mM sodium fluoride) containing 1 mM sodium vanadate, 10 μg/ml aprotinin, 10 μg/ml leupeptin, and 10 μg/ml pepstatin inhibitors. Cells were homogenized by passing the suspension through a 25-gauge needle (20 strokes). The lysate was centrifuged at 750×g for 10 min at 4° C. to pellet out the nuclei. The remaining supernatant was centrifuged for 30 min at 12,000×g. Protein was determined using the Lowry method and 50 μg of the lysate was used for the Western blot analysis. Proteins were resolved using the SDS-polyacrylamide gels and blotted onto nitrocellulose membranes. Membranes were washed with TBS (140 mM NaCl, 50 mM Tris-HCl, pH 7.2) containing 0.1% Tween 20 (TBST) and 5% skim milk to block the non-specific protein binding. Membranes were incubated with 1 μg/ml rabbit anti-iNOS polyclonal antibody (Abeam, Cambridge, Mass.), mouse anti-cyclin D1 antibody, mouse anti-cyclin E antibody (BD Biosciences, San Jose, Calif.) or rabbit anti-p27 antibody (Chemicon International, Temecula, Calif.) in TBST for overnight at 4° C., washed 5 times with TBST, and then incubated with goat anti-rabbit or rabbit anti-mouse IgG-horseradish peroxidase-conjugated secondary antibody (1:5,000) for 1.5 h at room temperature. The band was detected using the ECL method (Amersham Biosciences).

RT-PCR Analysis:

Following the treatments, medium was aspirated and 1 ml of TRIzol reagent (Invitrogen) was added and total RNA was extracted using the manufacturer's protocol. Five μg of RNA was used for the first strand cDNA synthesis using a first strand cDNA synthesis kit (Amersham Biosciences). Four μl of the cDNA mixture was used to amplify mRNA's of iNOS [(5′-CATGGCTTGCCCCTGGAAGTTTCT-3′, SEQ ID NO:1) and (5′-CCTCTATGGTGCCATCGGGCATC-3′, SEQ ID NO:2)], arginase I [(5′-CTCTAAGGGACAGCCTCGAGGA-3′, SEQ ID NO:3) and (5′-TGGGTTCACTTCCATGATATCTA-3′, SEQ ID NO:4)], arginase II [(5′-ATGTCCCTAAGGGGCAGCCTCTCGCGT-3′, SEQ ID NO:5) and (5′-CACAGCTGTAGCCATCTGACACAGCTC-3′, SEQ ID NO:6)], and eNOS [(5′-CCAGCTAGCCAAAGTCACCAT-3′, SEQ ID NO:7) and (5′-GTCTCGGAGCCATACAGGATT-3′, SEQ ID NO:8)].

Cell Cycle Analysis:

For DNA content analysis, harvested cells were centrifuged at 1,000×g for 5 min, fixed by the gradual addition of ice-cold 70% ethanol, and washed with PBS. Cells were then treated with RNase (10 μg/mL) for 30 min at 37° C., washed once with PBS, and resuspended and stained in 1 mL of 69 μmol/L propidium iodide in 38 mmol/L sodium citrate for 30 min at room temperature. The cell cycle phase distribution was determined by analytic DNA flow cytometry as described in (Vindelov L, et al. Methods Cell Biol, 33:127-37, 1990). The percentage of cells in each phase of the cell cycle was analyzed using a Modfit software (Verity Software House, Topsham, Me.).

Soft Agar Assay for Colony Formation:

After cells were treated with various conditions, they were seeded in six-well plates. The plates were first covered with phenol red-free MEM containing 0.6% agar and 10% FBS. The middle layer contained cells (5×10³) in phenol red-free MEM with 0.35% agar and 10% FBS. The top layer, consisting of the medium, was added to prevent drying of the agar in the plates. The plates were incubated for 21 days, after which the plates were stained in 0.5 ml of 0.005% crystal violet for 1 h and the cultures were inspected and photographed. The colony efficiency (CE) was determined by a count of the number of colonies greater than 15 mm in diameter, which was calculated as the average of colonies counted at 50× magnification in five individual fields manually (Liu S, et al. Oncogene, 23:1256-62, 2004).

Caspase-3 Like Proteolytic Activity:

Cells were washed twice in cold DPBS and lysed in buffer containing 10 mM Tris-HCl, 10 mM NaH₂PO₄/Na₂HPO₄ (pH 7.5), 130 mM NaCl, 1% Triton, and 10 mM sodium pyrophosphate. Cell lysate was incubated with a caspase-3 fluorogenic substrate N-acetyl-DEVD-7-amido-4-trifluoromethylcoumarin at 37° C. for 1 h. 7-Amido-4-trifluoromethylcoumarin liberated from the substrate was measured using a fluorescence plate reader (Perkin Elmer Life Sciences) with λₑₓ=400 nm and λₑₘ=505 nm (Wang S, et al. J Biol Chem, 279:25535-43, 2004). The fluorescence intensity was normalized to the protein levels measured with the Bradford protein assay kit (Sigma).

Measurement of Apoptosis by TUNEL Assay:

The terminal deoxynucleotidyl transferase-mediated nick-end labeling (TUNEL) assay was used for microscopic detection of apoptosis (Kotamraju S, et al. J Biol Chem, 277:17179-87, 2002). This assay is based on labeling of 3′ free hydroxyl ends of the fragmented DNA with fluorescein-dUTP catalyzed by terminal deoxynucleotidyl transferase. Procedures were followed according to a commercially available kit (ApoAlert) from Clontech. Apoptotic cells exhibit a strong nuclear green fluorescence that can be detected using a standard fluorescein filter (520 nm). All cells stained with propidium iodide exhibit a strong red cytoplasmic fluorescence at 620 nm. The apoptotic cells were detected by fluorescence microscopy equipped with rhodamine and FITC filters. The quantification of apoptosis was performed using the Metamorph image analysis package.

Statistical Analysis:

Results were analyzed by a one-way analysis of variance (ANOVA), and differences estimated by a Students t test were considered to be statistically significant at p<0.05.

Results

Statin Induce MCF-7 Cell Cytotoxicity.

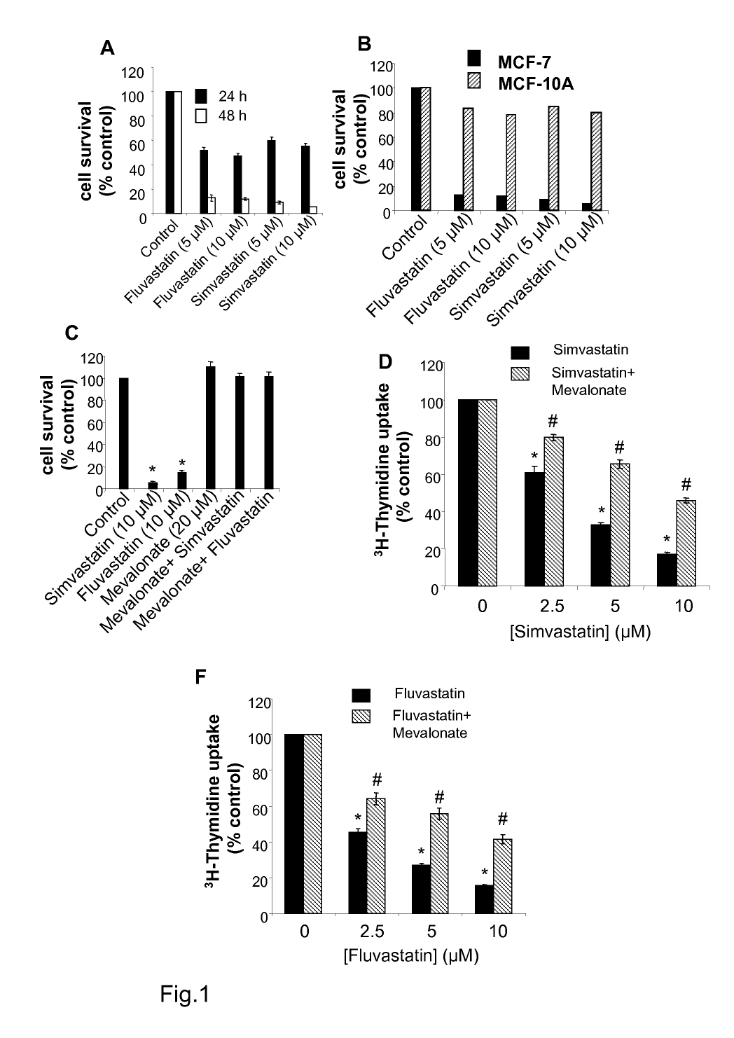

We assessed the effectiveness of simvastatin and fluvastatin to induce cytotoxicity in MCF-7 cells. MCF-7 breast cancer cells were treated with fluvastatin or simvastatin at different concentrations (0.5-20 μM) for 24-48 h. The changes in the number of viable cells were determined using the MTT assay that monitors the intracellular conversion of MTT to formazon spectrophotometrically max=562 nm). As shown in FIG. 1A, statins potently diminished the number of viable MCF-7 cells. Statins induced cytotoxicity in both MCF-7 breast cancer (malignant) cells (FIGS. 1A and B), and MDA-MB-231 (metastatic breast cancer cell lines) (data not shown). Fluvastatin and simvastatin did not affect non-cancerous mammary epithelial cells, MCF-10A (FIG. 1B). To determine whether statin-induced MCF-7 cell cytotoxicity is due to inhibition of HMG-CoA reductase activity, cells were pretreated with mevalonate prior to adding simvastatin and fluvastatin. Results show that mevalonate significantly reversed the cytotoxic effects of statins (FIG. 1C), suggesting that the HMG-CoA reductase activity (leading to cholesterol biosynthesis or protein isoprenylation) plays a pivotal role in statin-induced tumor cell cytotoxicity. However, pretreatment with squalene, an immediate precursor of cholesterol biosynthesis, did not prevent statin-induced cytotoxicity (data not shown). This suggests that modulation of isoprenylation of proteins may play a key role in statin-mediated effects in MCF-7 cells.

To further confirm the loss of cell proliferation (as detected by the MTT assay), we measured the DNA synthesis in MCF-7 cells treated with simvastatin or fluvastatin for a period of 24 h and monitored the uptake of ³H-thymidine during the last 3 h of the incubation. As seen in FIGS. 1D and E, both simvastatin and fluvastatin inhibited the uptake of ³H-thymidine that was partially reversed by mevalonate (FIGS. 1D and E).

Role of L-arginine Metabolizing Enzymes in Statin-Induced Cytotoxicity.

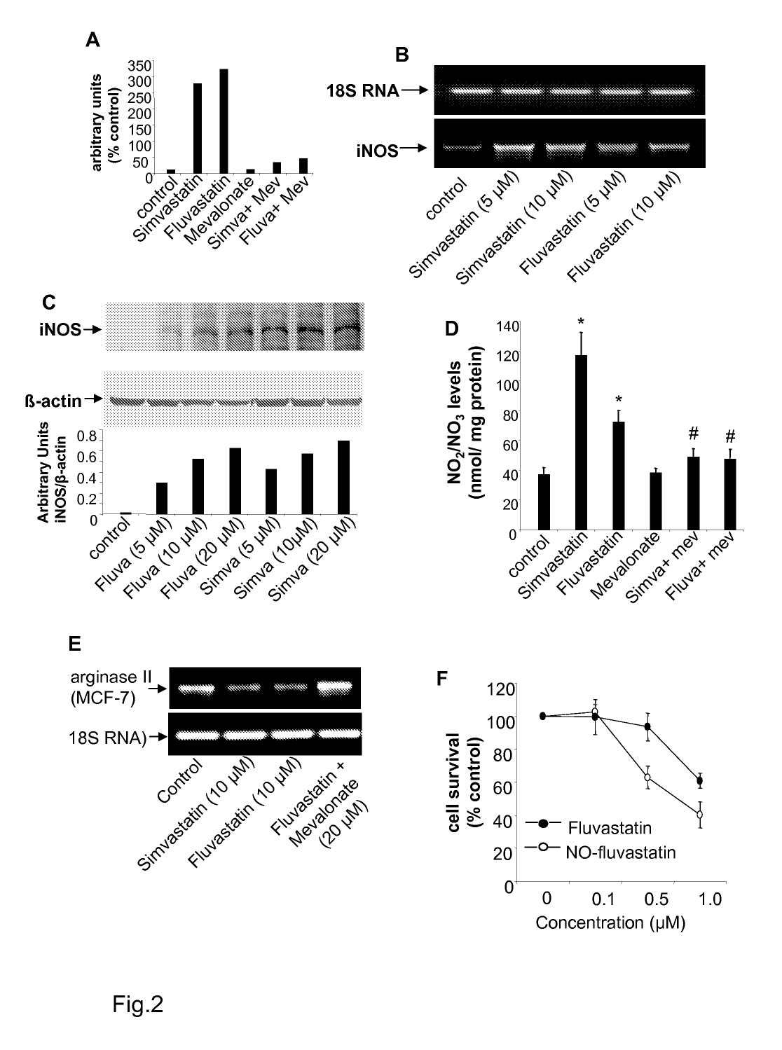

As statins are known to protect against endothelial dysfunction by modulating the nitric oxide synthase (NOS) and NO levels in endothelial cells (Kano H, et al. Biochem Biophys Res Commun, 259:414-9, 1999; Hernandez-Perera O, et al. J Clin Invest, 101:2711-9, 1998; Laufs U, et al. Circulation, 97:1129-35, 1998), we surmised that statins might also regulate NOS and NO levels in MCF-7 cells. To this end, we initially measured the DAF-2 derived green fluorescence. Both simvastatin and fluvastatin significantly increased NO-mediated DAF fluorescence in a dose-dependent manner (FIG. 2A: shown as % of control in arbitrary units). To identify the source of NO, we initially monitored the eNOS protein levels by Western blotting in MCF-7 cells treated with and without statins and found no detectable eNOS protein levels in control and treated MCF-7 cells (data not shown). However, quite unexpectedly, iNOS protein and iNOS mRNA levels were upregulated in cells treated with statins (FIGS. 2B and 2C). To further confirm that increased expression in iNOS protein corresponds to increased activity, we measured NO₂⁻/NO₃⁻ levels in the medium. Results indicate that statins increased NO₂⁻/NO₃⁻ levels (FIG. 2D). Mevalonate suppressed this increase in NO₂⁻/NO₃⁻ levels (FIG. 2D), suggesting that protein prenylation pathway (Rho or Ras GTPase) likely mediates iNOS expression and regulation. In addition to NOS-mediated oxidation of L-arginine to NO and citruline, L-arginine can also be metabolized by arginases to L-ornithine and urea within the urea cycle and is subsequently converted to polyamines (Morris S M Jr., J Nutrition 134: 2743S-2747S, 2004). Polyamines are known to increase cell proliferation (Chang C-I, et al. Cancer Res, 61:1100-1106, 2001). Since iNOS is significantly induced by statin treatment, it was of interest to measure the levels of arginases (Arg I and Arg II) in statin-treated MCF-7 cells. Arg I transcript levels could not be detected in MCF-7 cells but Arg II level was significantly down-regulated in statin-treated cells which was reversed by mevalonate (FIG. 2E). This result suggests a “crosstalk” between arginase and iNOS that plays a role in statin toxicity in MCF-7 cells. As statins increased .NO levels, we wondered whether N-nitroso-fluvastatin (NO-fluvastatin) supplementation in MCF-7 would be more effective in causing MCF-7 cell death as compared to fluvastatin alone. NO-fluvastatin is a hybrid molecule comprised of both statin and NO activities (Ongini E, et al. Proc Natl Acad Sci USA 101:8497-8502, 2004). Results show that NO-fluvastatin was more potent than fluvastatin alone in causing MCF-7 cells (FIG. 2F). This clearly implicates a major role for NO in statin-induced MCF-7 cell death.

Inhibition of Geranylgeranylation by Statins Induces iNOS Expression and Cell Death in MCF-7 Cells.

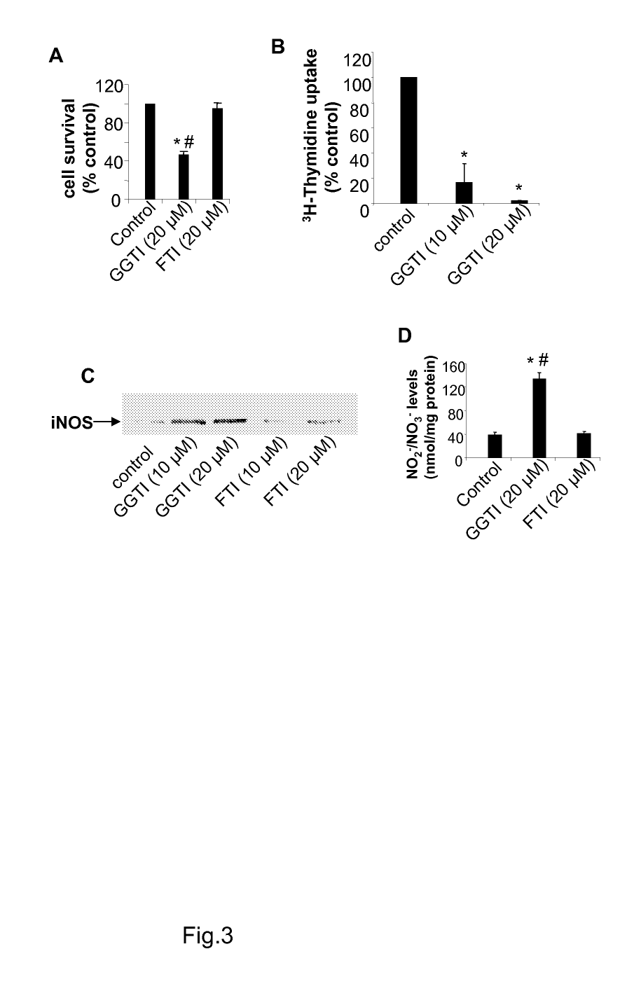

The present data showed that cholesterol-independent pathway is responsible for statin-induced effects. Statins have been reported to deplete the availability of prenylated substrates (Schafer W R, et al. Science, 245:379-85, 1989). Post-translational prenylation of small GTPases by the addition of a geranylgeranyl or farnesyl moiety is critical for cellular localization and signaling activity (Kaibuchi K, et al. Annu Rev Biochem, 68:459-86, 1999). To further confirm the involvement of isoprenoids on statin-induced, iNOS-dependent cell death, we investigated the effects of isoprenylation inhibitors. Pretreatment of MCF-7 cells with geranylgeranyltransferase inhibitor (GGTI-298), not farnesyltransferase inhibitor (FTI-277) induced MCF-7 cell death and loss of cell proliferation (FIG. 3A). The cell viability measurements were performed using the MTT assay and cell proliferation by monitoring the DNA synthesis using the ³H-thymidine uptake (FIG. 3B). To investigate whether inhibition of geranylgeranylation or farnesylation is responsible for enhanced iNOS expression, iNOS protein levels were measured in the presence of either GGTI-298 or FTI-277. As shown, GGTI and not FTI-277 dose-dependently induced the iNOS protein levels (FIG. 3C). Concomitantly, inhibition of geranygeranylation but not farnesylation increased the NO₂⁻/NO₃⁻ levels (FIG. 3D). Based on these results, we conclude that GGTI mimics the effects of statins, and therefore, it is likely that statin-mediated iNOS/NO induction and cytostatic/cytotoxic effects in MCF-7 cells occurs through geranylgeranylation of its downstream signaling targets (e.g., Rho or Rae GTPases).

Contrasting Effects of iNOS Inhibitor and iNOS Activator on Statin-Induced MCF-7 Cell Apoptosis:

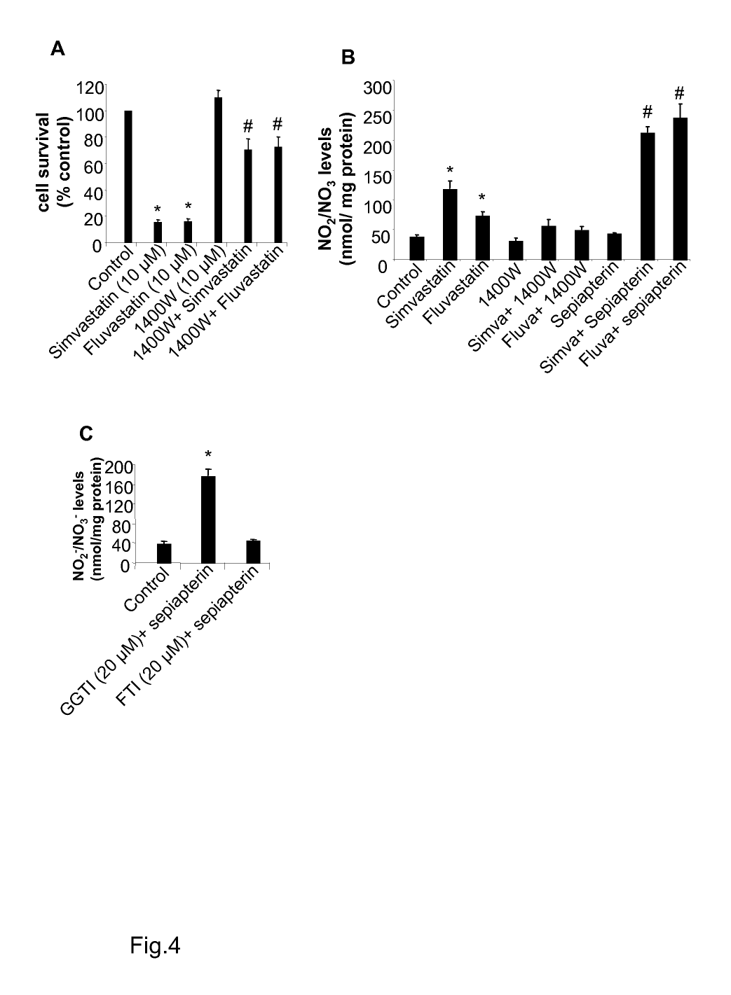

Pretreatment with 1400 W, a specific inhibitor of iNOS (Garvey E P et al., J Biol Chem 272, 4959-4963, 1997), partially reversed the statin-induced cell death/loss of proliferation in MCF-7 cells as measured by MTT reduction assay (FIG. 4A). Under these conditions, NO₂⁻/NO₃⁻ levels were decreased (FIG. 4B). Sepiapterin treatment significantly increased NO₂⁻/NO₃⁻ levels compared to statin alone treated conditions (FIG. 4B). Sepiapterin treatment alone in the absence of statin did not increase the NO2-/NO3- levels. Further verification that iNOS is involved in sepiapterin-induced NO was obtained from inhibition of geranylgeranylation and farnesylation. Treatment with GGTI-298 and sepiapterin significantly increased NO₂⁻/NO₃⁻ levels compared to GGTI-298 alone (FIG. 4C). In contrast, FTI-277 (farnesylation inhibitor) and sepiapterin had no effect on NO₂⁻/NO₃⁻ levels (FIG. 4C). Similar results were observed with respect to apoptosis as measured by the TUNEL assay. In the TUNEL assay, cells were treated with simvastatin or fluvastatin (5-10 μM) for 48 h in the presence or absence of 1400 W (10 μM), sepiapterin (50 μM) or mevalonate (20 μM) and stained for TUNEL-positive cells as an index of DNA fragmentation monitored by fluorescence microscopy (original magnification, ×100). Photographs were taken for the overlaid images of propidium iodide-and FITC-stained cells (TUNEL-positive cells). Yellow and red denote apoptotic and nonapoptotic cells, respectively. We observed that the TUNEL positive staining was enhanced in statin-treated MCF-7 cells. Sepiapterin (precursor of NOS co-factor, 5,6,7,8-BH4) treatment further augmented statin-induced TUNEL-positive cells. Pretreatment with 1400 W or mevalonate caused a decrease in the TUNEL positive cells. These results suggest that NO modulation may play a key role in decreasing or increasing the proapototic effects of statin in tumor cells.

The Effect of Statin on Cell Cycle Distribution—Role of NO:

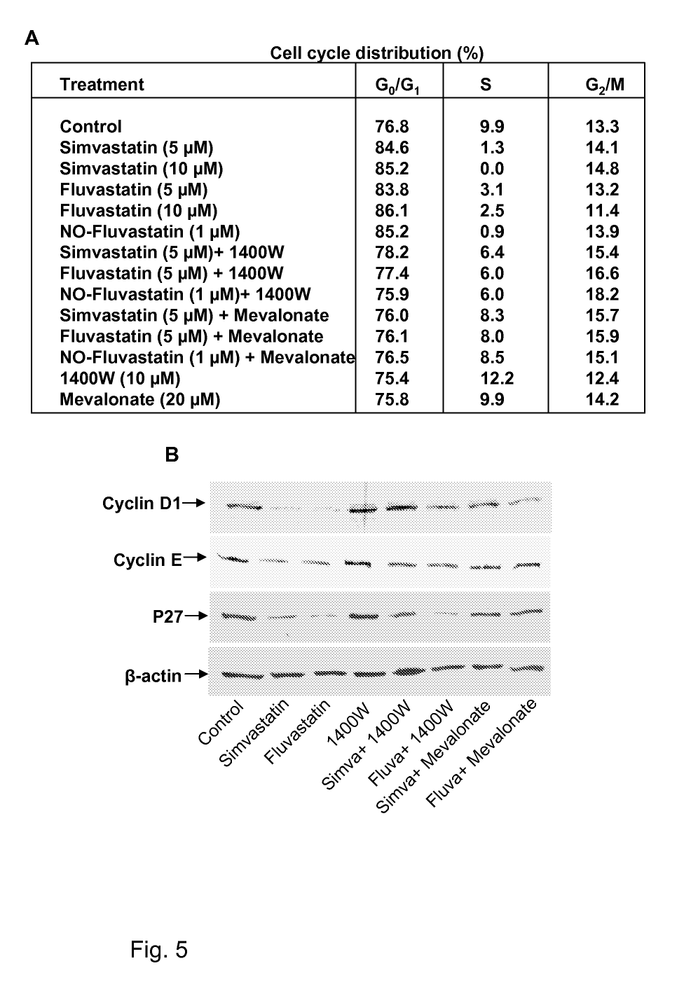

As NO has previously been reported to exert tumor cell cycle alterations (Pervin S, et al. Natl Acad Sci USA 98:3583-3588, 2001), we investigated the cytostatic effect of statins in MCF-7 cells. MCF-7 cells were treated with simvastatin and fluvastatin for 48 h in the presence and absence of 1400 W (10 μM) and mevalonate (20 μM). Cell cycle progression was examined using FACScan flow cytometry analysis. As shown in FIG. 5A (Table), both simvastatin and fluvastatin (5-10 μM) arrested MCF-7 cells in Go/G1 phase and as a result, the number of cells in the S phase was decreased. Similar effects were observed with NO-fluvastatin at a much lower concentrations (1 μM) as compared to native fluvastatin (Table). Statin-induced cell cycle alterations were partially reversed by the iNOS inhibitor (1400 W) and almost completely reversed by mevalonate (FIG. 5A, Table). As cell cycle progression from G0 to G2 phase involves activations of the cell regulatory proteins, cyclins D and E, we investigated the effects of statins and iNOS inhibitor on the cell cycle proteins. As expected, the cell cycle regulatory proteins, cyclin D1 and cyclin E (that are responsible for driving the cell cycle progression from Go/G1-S phase transition) were significantly decreased with statin treatments and restored in part by 1400 W or mevalonate. The levels of cyclin-dependent kinase inhibitor, p27, were also down-regulated by statin treatments (FIG. 5B). Therefore, under our experimental conditions, it appears that the decrease in cell cycle regulatory proteins is independent of the levels of cdk inhibitor(s) and possible, other regulatory mechanisms are involved.

Effects of Statins on Anchorage-Independent Growth of MCF-7 Cells:

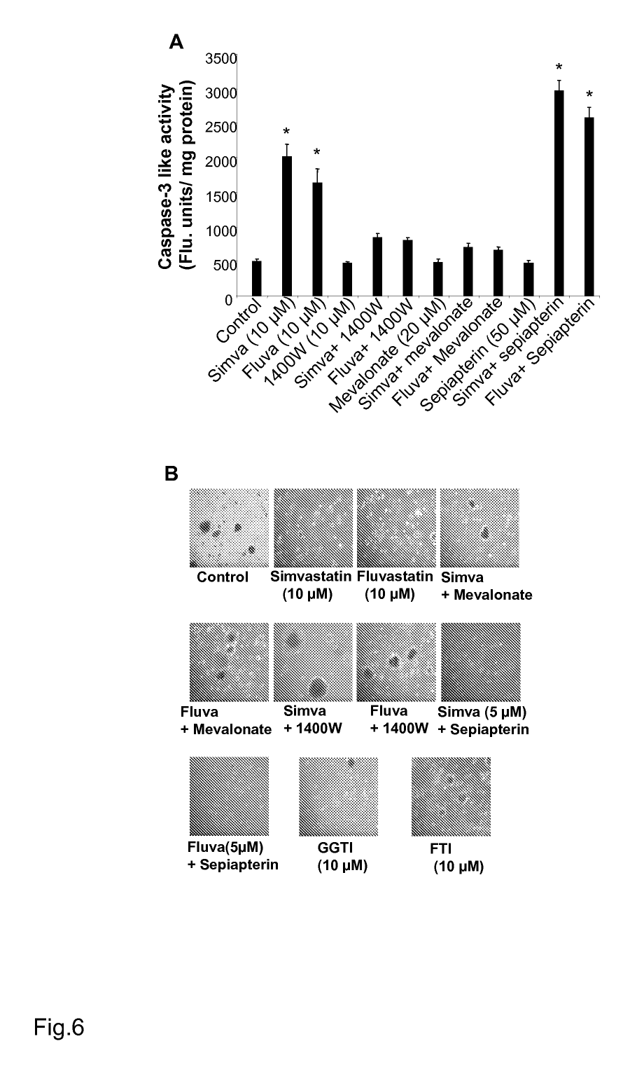

The long-term effects of statins and the inhibitors on the proliferation and survival of MCF-7 cells were determined using clonogenic assays (Ramanathan B, et al. Cancer Res, 65:8455-60, 2005). The extent of malignancy of cells corresponds to the attainment of anchorage-independent growth (Liu S, et al. Oncogene, 23:1256-62, 2004). MCF-7 cells were treated with simvastatin or fluvastatin in the presence or absence of either 1400 W or mevalonate or sepiapterin. In separate experiments, cells were treated with either GGTI-298 or FTI-277. At the end of the treatments, approximately 5×10³ cells were seeded onto a soft agar to determine their clonogenic efficiency after 21 days. Simvastatin and fluvastatin (10 μM) and GGTI-298 but not FTI-277 drastically lowered the visible colony formation in soft agar (FIG. 6B). In the presence of either 1400 W or mevalonate, the colony formation was restored in statin-treated cells (FIG. 6B). Sepiapterin supplementation completely inhibited the colony growth at a lower concentration of simvastatin or fluvastatin (5 μM) (FIG. 6B). Finally, these results indicate that statins are able to inhibit cell proliferation and anchorage-independent growth of MCF-7 cells by inhibiting geranylgeranylation, not farnesylation, through induction of nitric oxide mediated pathways.

Although the invention has been described in connection with specific embodiments in the above example, it is understood that the invention is not limited to such specific embodiments but encompasses all such modifications and variations apparent to a skilled artisan that fall within the scope of the appended claims.

We claim:

1. A method for treating breast cancer in a human or non-human animal comprising the step of:
administering to a human or non-human animal in need of said treatment a first agent selected from a hydroxymethylglutaryl coenzyme A (HMG-CoA) reductase inhibitor, an HMG-CoA reductase inhibitor coupled with a nitric oxide molecule, a protein geranylgeranyl transferase (GGTase) inhibitor, and a GGTase inhibitor coupled with a nitric oxide molecule and a second agent that promotes inducible nitric oxide synthase (iNOS)-catalyzed nitric oxide formation wherein the amount of the first agent and the amount of the second agent are therapeutically effective.

2. The method of claim 1, wherein a human breast cancer patient is treated.

3. The method of claim 1, wherein the first agent is an HMG-CoA reductase inhibitor.

4. The method of claim 3, wherein the HMG-CoA reductase inhibitor is selected from lovastatin, simvastatin, pravastatin, fluvastatin, atorvastatin, mevastatin, cerivastatin, pitavastatin, rosuvastatin, compactin, dalvastatin, and fluindostatin.

5. The method of claim 3, wherein the HMG-CoA reductase inhibitor is a hydrophobic HMG-CoA reductase inhibitor selected from lovastatin, simvastatin, fluvastatin, atorvastatin, mevastatin, cerivastatin, pitavastatin, rosuvastatin, compactin, and dalvastatin.

6. The method of claim 5, wherein the HMG-CoA reductase inhibitor is selected from simvastatin and fluvastatin.

7. The method of claim 1, wherein the first agent is a geranylgeranyl transferase inhibitor.

8. The method of claim 1, wherein the second agent is selected from tetrahydrobiopterin (BH₄), a synthetic NOS activator, a compound that can be converted to BH₄ inside a cell, a compound that facilitates the regeneration of BH₄ inside a cell, L-arginine, a compound that can be converted to L-arginine inside a cell, an arginase inhibitor, and a compound that can increase the metabolism of asymmetric dimethyl-arginine (ADMA).

9. The method of claim 8, wherein the synthetic NOS activator is a pteridine derivative.

10. The method of claim 8, wherein the synthetic NOS activator is 6-methyltctrahydropterin.

11. The method of claim 8, wherein the compound that can be converted to BH₄ is selected from sepiapterin, BH₂, 7,8-dihydroneopterin triphosphate, and 6-pyruvoyl-tetrahydropterin.

12. The method of claim 11, wherein the compound is sepiapterin.

13. The method of claim 8, wherein the compound that facilitates the regeneration of BH₄ is selected from folic acid and folate.

14. The method of claim 13, wherein the folate is 5-methyltetrahydrofolate.

15. The method of claim 8, wherein the second agent is L-arginine.

16. The method of claim 8, wherein the second agent is an arginase inhibitor.

17. A method for treating breast cancer in a human or non-human animal comprising the step of:
administering to a human or non-human animal in need of said treatment an agent selected from an HMG-CoA reductase inhibitor coupled with a nitric oxide molecule and a GGTase inhibitor coupled with a nitric oxide molecule wherein the amount of the agent is therapeutically effective.

18. A method for inhibiting the proliferation or causing the death of breast cancer cells of a human or non-human animal comprising the step of:
exposing the breast cancer cells to a first agent selected from a hydroxymethylglutaryl coenzyme A (HMG-CoA) reductase inhibitor, an HMG-CoA reductase inhibitor coupled with a nitric oxide molecule, a protein geranylgeranyl transferase (GGTase) inhibitor, and a GGTase inhibitor coupled with a nitric oxide molecule and a second agent that promotes inducible nitric oxide synthase (iNOS)-catalyzed nitric oxide formation wherein the amount of the first agent and the amount of the second agent are sufficient to inhibit the proliferation or cause the death of the breast cancer cells.

19. A composition comprising a first agent selected from a hydroxymethylglutaryl coenzyme A (HMG-CoA) reductase inhibitor, an HMG-CoA reductase inhibitor coupled with a nitric oxide molecule, a protein geranylgeranyl transferase (GGTase) inhibitor, and a GGTase inhibitor coupled with a nitric oxide molecule and a second agent that promotes inducible nitric oxide synthase (iNOS)-catalyzed nitric oxide formation wherein the amount of the first agent and the amount of the second agent are therapeutically effective for treating breast cancer.

20. A kit comprising:
a first agent selected from a hydroxymethylglutaryl coenzyme A (HMG-CoA) reductase inhibitor, an HMG-CoA reductase inhibitor coupled with a nitric oxide molecule, a protein geranylgeranyl transferase (GGTase) inhibitor, and a GGTase inhibitor coupled with a nitric oxide molecule;
a second agent that promotes inducible nitric oxide synthase (iNOS)-catalyzed nitric oxide formation; and
an instruction manual on administering the first agent and the second agent to treat breast cancer, wherein the amount of the first agent and the amount of the second agent are sufficient for treating breast cancer.
